SEQUENCE LISTING (2 sequences)

SEQ ID NO: 1 - DNA, 91 bp, Artificial Sequence
  Oligonucleotide
aagcagtggtatcaacgcagagtacttttttttttttttttttttttttttttttttttttttttttttttttttttttttttttttttvn

SEQ ID NO: 2 - DNA, 39 bp, Artificial Sequence
  Oligonucleotide
aagcagtggtatcaacgcagagtggccatattggccggg

(19) United States
(12) Patent Application Publication
(10) Pub. No.: US 2011/0236345 A1
(43) Pub. Date: Sep. 29, 2011

(54) COMPOSITION AND METHODS FOR ELICITING AN IMMUNE RESPONSE

(75) Inventors: John H. Sampson, Raleigh, NC (US); Duane A. Mitchell, Daleigh, NC (US)

(73) Assignee: Duke University, Durham, NC (US)

(21) Appl. No.: 12/999,118

(22) PCT Filed: Jul. 1, 2009
(86) PCT No.: PCT/US09/49391

§ 371 Date: Jun. 16, 2011

Related U.S. Application Data
(60) Provisional application No. 61/077,922, filed on Jul. 3, 2008.

(51) Int. Cl. A61K 38/20; A61K 39/00; A61P 35/00; A61P 37/04

(52) U.S. Cl. 424/85.2; 424/277.1

(57) ABSTRACT

The present invention relates to compositions, methods, and kits for eliciting an immune response, in particular for eliciting an immune response to at least one antigen expressed by a cancer cell, in particular for treating and preventing cancer.

### CROSS REFERENCE TO RELATED APPLICATION

This application claims priority under 35 USC §119 to U.S. Provisional Application No. 61/077,922, filed Jul. 3, 2008, which is herein incorporated by reference in its entirety.

### FIELD OF THE INVENTION

The present invention relates to compositions, methods, and kits for eliciting an immune response, in particular for eliciting an immune response to at least one antigen expressed by a cancer cell.

### BACKGROUND OF THE INVENTION

Methods for treating cancers include the use of chemotherapeutics, radiation therapy, and surgery. T he identification of a number of tumor antigens has led to attempts at developing cell-based therapies. Some methods have relied on first identifying a tumor antigen, i.e., a polypeptide that is expressed preferentially in tumor cells, relative to non-tumor cells. For example, several human tumor antigens have been isolated from melanoma patients, and identified and characterized.

Despite aggressive multi-modality therapy including surgery, radiation, and chemotherapy, the prognosis for patients with cancer remains relatively poor. Moreover, the non-specific nature of conventional therapy for cancer often results in incapacitating damage to surrounding normal and systemic tissues. Thus, there is a need for the development of effective therapeutic and prophylactic strategies that target tumor cells.

### SUMMARY OF THE INVENTION

In one aspect, the present invention provides a method for eliciting in a subject an immune response to at least one antigen expressed by a cancer cell. The method comprises administering to the subject a pharmaceutically acceptable composition comprising a plurality of developmental cell antigens, or nucleic acids encoding the plurality of developmental cell antigens, wherein the pharmaceutically acceptable composition, when administered to the subject, elicits the immune response to the at least one antigen.

In another aspect, the present invention provides a pharmaceutically acceptable composition comprising a plurality of antigens expressed by a developmental cell, or nucleic acids encoding the plurality of antigens. The pharmaceutically acceptable composition, when administered to a subject, elicits an immune response against at least one antigen expressed by a cancer cell.

In other aspects, the present invention provides a prophylactically or therapeutically effective amount of a pharmaceutically acceptable composition, wherein the pharmaceutically acceptable composition comprises a plurality of antigens expressed by a developmental cell, or nucleic acids encoding the plurality of antigens, wherein the pharmaceutically acceptable composition, when administered to a subject, elicits an immune response against at least one antigen expressed by a cancer cell.

In one aspect, the present invention provides a method for eliciting in a subject an immune response to at least one antigen expressed by a cancer cell. The method comprises administering to the subject a composition comprising an effective amount of antigen presenting cells, T-lymphocytes, or both, wherein the antigen presenting cells and T lymphocytes have been sensitized in vitro with a sensitizing-effective amount of a plurality of antigens expressed by a developmental cell, wherein the effective amount of antigen presenting cells, T lymphocytes, or both is sufficient to elicit the immune response to the at least one antigen.

In some aspects, the present invention provides a method for making cancer antigen-primed antigen-presenting cells, the method comprising contacting antigen-presenting cells with a plurality of developmental cell antigens, or nucleic acids encoding the plurality of developmental cell antigens, in vitro under a condition sufficient for the plurality of developmental cell antigens to be presented by the antigen-presenting cells.

In one aspect, the present invention provides a composition comprising antigen-presenting cells contacted with a plurality of developmental cell antigens, or nucleic acids encoding the plurality of developmental cell antigens, in vitro under a condition sufficient for the plurality of developmental cell antigens to be presented by the antigen-presenting cells.

In other aspects, the present invention provides a method for making cancer antigen-specific lymphocytes. The method comprises:

a) contacting antigen-presenting cells with a plurality of developmental cell antigens, or nucleic acids encoding the plurality of developmental cell antigens, in vitro under a condition sufficient for the plurality of developmental cell antigens to be presented by the antigen-presenting cells; and

b) contacting lymphocytes with the antigen-presenting cells of step a) under conditions sufficient to produce cancer antigen-specific lymphocytes capable of eliciting an immune response against a cancer cell.

In one aspect, the present invention provides a composition comprising T lymphocytes contacted with antigen-presenting cells under conditions sufficient to produce cancer antigen-specific lymphocytes capable of eliciting an immune response against a cancer cell, wherein the antigen-presenting cells have been contacted with a plurality of developmental cell antigens, or nucleic acids encoding the plurality of developmental cell antigens, in vitro under a condition sufficient for the plurality of developmental cell antigens to be presented by the antigen-presenting cells.

In another aspect, the present invention provides a method for treating or preventing cancer in a subject, the method comprising administering to the subject a therapeutically or prophylactically effective amount of a composition as described herein.

In some aspects, the present invention provides a method for eliciting in a subject an immune response to at least one antigen expressed by a cancer cell, the method comprising administering to the subject a pharmaceutically acceptable composition comprising dendritic cells loaded ex vivo with a plurality of developmental cell antigens, or nucleic acids encoding the plurality of developmental cell antigens, wherein the pharmaceutically acceptable composition, when administered to the subject, elicits an immune response to the at least one antigen.

In other aspects, the present invention provides a method of eliciting in a subject an immune response to a cancer cell, the method comprising administering to the subject a pharmaceutically acceptable composition comprising antibodies raised against a plurality of developmental cell antigens.

In another aspect, a kit is provided in accordance with the present invention. The kit comprises a pharmaceutically acceptable composition comprising a plurality of antigens expressed by a developmental cell, or nucleic acids encoding the plurality of antigens, wherein the pharmaceutically acceptable composition, when administered to a subject, elicits an immune response against at least one antigen expressed by a cancer cell.

### BRIEF DESCRIPTION OF THE DRAWINGS

FIG. 1 is a graph showing the effect on percent survival of mice with malignant astrocytoma vaccinated with total tumor RNA.

FIG. 2 is a graph comparing the effect on percent survival of mice with malignant glioma vaccinated with total embryonic RNA or total tumor RNA.

FIG. 3 is an agarose gel electrophoresis showing (a) amplification products; and (b) in vitro transcribed RNA.

FIG. 4 is a graph showing enrichment of cancer stem cell antigens in CD133(+) astrocytomas using subtractive hybridization with CD133(−) RNA.

### DETAILED DESCRIPTION OF THE INVENTION

Applicants have discovered that cancer cells in a subject, in particular cancer stem cells can be immunologically targeted by using a cohort of antigens expressed by a developmental cell, or by using nucleic acids encoding the antigens. Tumor development resembles a reversion to a primitive state of tissue differentiation reminiscent of the proliferative and invasive state of cells such as embryonic cells during fetal development. Thus, certain developmentally regulated antigens, which can also be expressed in a wide variety of tumors, can serve as universal tumor rejection antigens, for example in immunotherapy against malignancy. The invention can thus circumvent the need to isolate and identify a tumor antigen.

For example, the invention is applicable, but not limited, to the development of therapies for brain cancers (e.g., gliomas), lung cancers, liver cancers, cervical cancers, soft tissue sarcomas, endocrine tumors, hematopoietic cancers, melanomas, bladder cancers, breast cancers, pancreatic cancers, prostate cancers, colon cancers, and ovarian cancers.

### I. Definitions

The term “immune response” refers herein to any response to an antigen or antigenic determinant by the immune system. Exemplary immune responses include humoral immune responses (e.g. production of antigen-specific antibodies (neutralizing or otherwise)) and cell-mediated immune responses (e.g. lymphocyte proliferation).

The term “developmental cell” refers herein to embryonic cells, embryonic stem cells (ESC), fetal cells, fetal stem cells, placenta cells, placenta progenitor cells, umbilical cord stem cells, embryonic-like cells from umbilical cord blood, and postnatal, tissue-specific progenitor cells. The “developmental cell” can be a primary cell or a cell line obtained therefrom, including stable cell lines obtained with or without proliferation, transformation, cloning, and/or any other type of manipulation.

The term “developmental cell antigens” refers herein to antigens corresponding to antigens expressed by a developmental cell.

### II. Methods and Compositions

In one aspect, the present invention provides a method for eliciting in a subject an immune response to at least one antigen expressed by a cancer cell. The method comprises administering to the subject a pharmaceutically acceptable composition comprising a plurality of developmental cell antigens, or nucleic acids encoding the plurality of developmental cell antigens, wherein the composition, when administered to the subject, elicits an immune response to the at least one antigen.

Generally, the immune response can include the humoral immune response, the cell-mediated immune response, or both. For example, antigen presentation through an immunological pathway involving MHC II proteins or direct B-cell stimulation can produce a humoral response; and, antigens presented through a pathway involving MHC I proteins can elicit the cellular arm of the immune system.

A humoral response can be determined by a standard immunoassay for antibody levels in a serum sample from the subject receiving the pharmaceutically acceptable composition. A cellular immune response is a response that involves T cells and can be determined in vitro or in vivo. For example, a general cellular immune response can be determined as the T cell proliferative activity in cells (e.g., peripheral blood leukocytes (PBLs)) sampled from the subject at a suitable time following the administering of the pharmaceutically acceptable composition. Following incubation of e.g., peripheral blood mononuclear cells (PBMC) with a stimulator for an appropriate period, [³H]thymidine incorporation can be determined. The subset of T cells that is proliferating can be determined using flow cytometry. T cell cytotoxicity (CTh) can also be determined.

In one embodiment, the immune response that is elicited is sufficient for prophylactic or therapeutic treatment of a neoplastic disease or a symptom associated therewith, particularly cancer (e.g., a tumor). Accordingly, a beneficial effect of the pharmaceutically acceptable composition will generally at least in part be immune-mediated, although an immune response need not be positively demonstrated in order for the compositions and methods described herein to fall within the scope of the present invention.

Administering to both human and non-human vertebrates is contemplated within the scope of the present invention. Veterinary applications also are contemplated. Generally, the subject is any living organism in which an immune response can be elicited. Examples of subjects include, without limitation, humans, livestock, dogs, cats, mice, rats, and transgenic species thereof.

The subject can either have a neoplastic disease (e.g., a tumor), or be at risk of developing the neoplastic disease. Subjects can be characterized by clinical criteria, for example, those with advanced neoplastic disease or high tumor burden exhibiting a clinically measurable tumor. A clinically measurable tumor is one that can be detected on the basis of tumor mass (e.g., by palpation, MRI, CAT scan, X-ray). Thus, for example, the pharmaceutically acceptable composition in accordance with the present invention can be administered to subjects with advanced disease with the objective of mitigating their condition. Preferably, a reduction in tumor mass occurs as a result of administering the pharmaceutically acceptable composition of the present invention, but any clinical improvement constitutes a benefit. Clinical improvement includes decreased risk or rate of progression or reduction in pathological consequences of a tumor, for example.

By way of another example, the subject can be one that has a history of cancer and has been responsive to another mode of therapy. The other therapy may have included e.g., surgical resection, radiotherapy, chemotherapy, and other modes of immunotherapy whereby as a result of the other therapy, the subject presents no clinically measurable tumor. However, the subject can be one determined to be at risk for recurrence or progression of the cancer, either near the original tumor site, or by metastases. Such subjects can be further categorized as high-risk and low-risk subjects. The subdivision can be made on the basis of features observed before or after the initial treatment. These features are known in the clinical arts, and are suitably defined for each different cancer. Features typical of high risk subgroups are those in which the tumor has invaded neighboring tissues, or which show involvement of lymph nodes. Thus, for example, a pharmaceutically acceptable composition of the present invention can be administered to the subject to elicit an anti-cancer response primarily as a prophylactic measure against recurrence. Preferably, administering the composition delays recurrence of the cancer, or more preferably, reduces the risk of recurrence (i.e., improves the cure rate). Such parameters can be determined in comparison with other patient populations and other modes of therapy.

The pharmaceutically acceptable composition can be administered at any time that is appropriate. For example, the administering can be conducted before or during traditional therapy of a subject having a tumor burden, and continued after the tumor becomes clinically undetectable. The administering also can be continued in a subject showing signs of recurrence.

The cancer cell can be of any type of cancer including, but not limited to, brain cancers (e.g., gliomas), lung cancers, liver cancers, cervical cancers, soft tissue sarcomas, endocrine tumors, hematopoietic cancers, melanomas, bladder cancers, breast cancers, pancreatic cancers, prostate cancers, colon cancers, ovarian cancers, skin cancers, and combinations thereof. The cancer also can be characterized as benign or malignant. In one embodiment, the cancer is a high grade glioma. In another embodiment, the high grade glioma is a glioblastoma multiforme, an anaplastic astrocytoma, or an oligodendroglioma.

The pharmaceutically acceptable composition can be administered in a therapeutically or a prophylactically effective amount, wherein the pharmaceutically acceptable composition comprises the plurality of developmental cell antigens, or nucleic acids encoding the plurality of developmental cell antigens, either alone or in combination with one or more other antigens. Administering the pharmaceutically acceptable composition of the present invention to the subject can be carried out using known procedures, and at dosages and for periods of time sufficient to achieve a desired effect. For example, a therapeutically or prophylactically effective amount of the pharmaceutically acceptable composition, can vary according to factors such as the age, sex, and weight of the subject. Dosage regima can be adjusted by one of ordinary skill in the art to elicit the desired immune response including immune responses that provide therapeutic or prophylactic effects.

The pharmaceutically acceptable composition can be administered to the subject at any suitable site, for example a site that is distal to or proximal to a primary tumor. The route of administering can be parenteral, intramuscular, subcutaneous, intradermal, intraperitoneal, intranasal, intravenous (including via an indwelling catheter), via an afferent lymph vessel, or by any other route suitable in view of the neoplastic disease being treated and the subject's condition. Preferably, the dose will be administered in an amount and for a period of time effective in bringing about a desired response, be it eliciting the immune response or the prophylactic or therapeutic treatment of the neoplastic disease or symptoms associated therewith.

The pharmaceutically acceptable composition can be given subsequent to, preceding, or contemporaneously with other therapies including therapies that also elicit an immune response in the subject. For example, the subject may previously or concurrently be treated by chemotherapy (e.g., by an alkylating agent such as temozolomide), radiation therapy, and other forms of immunotherapy, such other therapies preferably provided in such a way so as not to interfere with the immunogenicity of the compositions of the present invention.

Administering can be properly timed by the care giver (e.g., physician, veterinarian), and can depend on the clinical condition of the subject, the objectives of administering, and/or other therapies also being contemplated or administered. In some embodiments, an initial dose can be administered, and the subject monitored for either an immunological or clinical response, preferably both. Suitable means of immunological monitoring include using patient's peripheral blood lymphocyte (PBL) as responders and neoplastic cells as stimulators. An immunological reaction also can be determined by a delayed inflammatory response at the site of administering. One or more doses subsequent to the initial dose can be given as appropriate, typically on a monthly, semimonthly, or preferably a weekly basis, until the desired effect is achieved. Thereafter, additional booster or maintenance doses can be given as required, particularly when the immunological or clinical benefit appears to subside.

A number of developmentally regulated proteins expressed during fetal development and within embryonic stem cells are conserved amongst mammalian species (and in some cases, even in Drosophila) and exhibit a high degree of homology at the protein and nucleic acid level with humans. Accordingly, xenogeneic embryonic tissue antigens and embryonic stem cell antigens are examples of antigens that can represent suitable alternatives to human embryonic antigens for use in accordance with the present invention. In one embodiment, the plurality of developmental cell antigens are xenogeneic, allogeneic, or autologous to the subject. In another embodiment, the plurality of developmental cell antigens are xenogeneic to the subject, the plurality of developmental cell antigens corresponding to antigens expressed by a developmental cell xenogeneic to the subject.

As further illustrated below, the pharmaceutically acceptable composition can comprise the plurality of developmental cell antigens or nucleic acids encoding the plurality of developmental cell antigens.

### A. Nucleic Acids

Generally, the subject can be inoculated with the pharmaceutically acceptable composition comprising nucleic acids through any parenteral route. For example, the subject can be inoculated by intravenous, intraperitoneal, intradermal, subcutaneous, inhalation, or intramuscular routes, or by particle bombardment using a gene gun. Preferably, muscle tissue can be a site for the delivery and expression of polynucleotides. A dose of polynucleotides can be administered into muscle by multiple and/or repetitive injections, for example, to extend administration over long periods of time. Thus, muscle cells can be injected with polynucleotides coding for the plurality of developmental cell antigens, and the expressed antigens can be presented by muscle cells in the context of antigens of the major histocompatibility complex to elicit the immune response against the developmental cell antigens.

The epidermis can be another useful site for the delivery and expression of polynucleotides, for example either by direct injection or particle bombardment. A dose of polynucleotides can be administered in the epidermis, for example by multiple injections or bombardments to extend administering over long periods of time. For example, skin cells can be injected with polynucleotides coding for the plurality of developmental cell antigens, and the expressed antigens can be presented by skin cells in the context of antigens of the major histocompatibility complex to elicit the immune response against the developmental cell antigens.

A subject also can be inoculated by a mucosal route. The polynucleotides can be administered to a mucosal surface by a variety of methods including polynucleotide-containing nose-drops, inhalants, suppositories, microsphere-encapsulated polynucleotides, or by bombardment with polynucleotide-coated gold particles. For example, the nucleic acids coding for the plurality of developmental cell antigens can be administered to a respiratory mucosal surface.

Any appropriate physiologically compatible medium, such as saline for injection, or gold particles for particle bombardment, is suitable for introducing polynucleotides into a subject.

### 1. RNA

In some embodiments, the pharmaceutically acceptable composition comprises nucleic acids encoding the plurality of developmental cell antigens. In one embodiment, the nucleic acids encoding the plurality of antigens comprise RNAs, for example cell total RNA and/or poly A⁺ RNA. The RNAs comprise translatable RNA templates to guide the intracellular synthesis of amino acid chains that provide the plurality of developmental cell antigens. RNAs encoding the plurality of developmental cell antigens also can be in vitro transcribed, e.g., reverse transcribed to produce cDNAs that can then be amplified by PCR, if desired, and subsequently transcribed in vitro, with or without cloning the cDNA.

Also included are RNAs that are provided as a fractionated preparation with respect to a non-RNA component(s) in order to decrease the concentration of the non-RNA components, such as proteins, lipids, and/or DNA, and, thereby, enriching the preparation for RNA. Thus, when the RNAs are provided from a preparation comprising RNAs and non-RNA components, the preparation can be fractionated or otherwise treated to decrease the concentration of proteins, lipids, and/or DNA, if present, in the preparation, and enrich the preparation for RNA. For example, RNA purification methods known to one of ordinary skill in the art can be used to at least partially purify the RNAs. Optionally, the RNA preparation is treated with a protease or RNase-free DNase.

Optionally, an RNA preparation also can be fractionated with respect to RNA (e.g., by subtractive hybridization) such that, for example, cancer stem cell-specific RNAs are provided. Antigens expressed within the stem cell fraction of tumor cells, which can share expression profiles with the developmental cell, can be effectively targeted by this approach. Cancer stem cells can be more resistant to standard therapies and can contribute to tumor recurrence. Accordingly, a technique such as subtractive hybridization can be utilized to enrich for antigens expressed specifically in cancer stem cell subsets and, therefore, subtractive hybridization can be used to provide an RNA preparation that is devoid of RNAs encoding antigens expressed in normal postpartum tissues, for example. Such fractionation can provide RNAs encoding shared developmental cell antigens expressed in malignant cells by removal of at least a portion of RNAs encoding antigens that may be expressed in normal tissues.

Thus, for example, subtractive hybridization using RNAs from normal adult tissue can be performed. Accordingly, in one embodiment, the nucleic acids encoding the plurality of developmental cell antigens are enriched for RNA transcripts that are not transcribed in an adult cell or, if transcribed in the adult cell, are transcribed at a substantially reduced level compared to a developmental cell.

A number of methods are available to one of ordinary skill in the art to prepare RNAs encoding the plurality of developmental cell antigens. For example, a developmental cell preparation comprising released total RNAs can be prepared by sonicating developmental cells in a mammalian cell culture medium such as Opti-MEM® or a buffer such as phosphate buffered saline. Other methods for disrupting cells also are suitable, provided that the method does not completely degrade the RNAs. For example, RNAs also can be prepared by employing conventional RNA purification methods such as guanidinium isothiocyanate methods and/or oligo dT chromatography methods for isolating poly A⁺ RNA. Also, for example, RNAs prepared from developmental cells can be reverse transcribed into cDNAs, which then can be amplified by conventional PCR techniques to provide an essentially unlimited supply of cDNAs corresponding to the RNAs encoding the antigens. Conventional in vitro transcription techniques and bacterial polymerases can be used to produce in vitro transcribed RNAs, or the in vitro transcribed RNAs can be synthesized from cloned DNA sequences encoding the plurality of developmental cell antigens.

### 2. DNA

In another embodiment, the nucleic acids encoding the plurality of developmental cell antigens comprise DNAs having open reading frames encoding the plurality of developmental cell antigens. For example, a pharmaceutically acceptable composition comprising expression vectors having DNA open reading frames encoding the plurality of developmental cell antigens can be administered to the subject.

Genomic DNA fragments and/or cDNAs comprising open reading frames encoding the plurality of developmental cell antigens can be employed in the methods of the present invention. cDNAs can be prepared from the above-described RNAs coding for the plurality of developmental cell antigens using techniques known to one of ordinary skill in the art.

DNA can be fragmented, for example by physical fragmentation or, preferably, by enzymatic cleavage, i.e. use of restriction endonucleases. Fragmentation methods are well known to those skilled in the art and can be varied (e.g., by use of different restriction endonucleases or combinations and digestion times) to obtain fragments differing in size and composition. DNAs or fragments thereof having open reading frames encoding the plurality of developmental cell antigens can be cloned into expression vectors by methods and reagents known in the art. For example, a pharmaceutically acceptable composition comprising a library of expression vectors or a sub-library thereof can be administered to the subject.

Preparation of a DNA expression library can be performed by well known methods. Standard cloning vectors can be employed that have a selectable marker (e.g., ampicillin) and, preferably an origin of replication (e.g., ori) and a suitable promoter. Bacteria (e.g., E. coli) or other suitable host can then transformed with the vectors, and transformants cultured by standard procedures and the plasmid DNA isolated by such methods as chromatographic or organic separation. For example, plasmids are available for cloning into a site which can direct the plurality of antigens expressed by the open reading frames to MHC I or II. Expression vectors used for eliciting an immune response and methods of using same are described in U.S. Patent Application Publication No. 20040241140, which is incorporated herein for its teaching of expression vectors used for eliciting an immune response and methods of using same.

When taken up by a cell (e.g., muscle cell, antigen presenting cell (APC) such as a dendritic cell, macrophage, etc.), a DNA molecule can be present in the cell as an extrachromosomal molecule and/or can integrate into the chromosome. DNA can be introduced into cells in the form of a plasmid which can remain as separate genetic material. Alternatively, linear DNAs that can integrate into the chromosome can be introduced into the cell. Optionally, when introducing DNA into a cell, reagents which promote DNA integration into chromosomes can be added.

Thus, preferably DNAs include regulatory elements necessary for expression of an open reading frame. Such elements can include, for example, a promoter, an initiation codon, a stop codon, and a polyadenylation signal. In addition, enhancers can be included. As is known in the art, these elements are preferably operably linked to a sequence that encodes a developmental cell antigen. Regulatory elements are preferably selected that are operable in the species of the subject to which they are to be administered. Initiation codons and stop codons in frame with a coding sequence are preferably included.

Examples of promoters include but are not limited to promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, Cytomegalovirus (CMV) such as the CMV immediate early promoter, Epstein Barr Virus (EBV), Rous Sarcoma Virus (RSV) as well as promoters from human genes such as human actin, human myosin, human hemoglobin, human muscle creatine, and human metalothionein. Examples of suitable polyadenylation signals include but are not limited to SV40 polyadenylation signals and LTR polyadenylation signals.

In addition to the regulatory elements required for DNA expression, other elements may also be included in the DNA molecule. Such additional elements include enhancers. Enhancers include the promoters described hereinabove. Preferred enhancers/promoters include, for example, human actin, human myosin, human hemoglobin, human muscle creatine and viral enhancers such as those from CMV, RSV and EBV.

Optionally, the DNAs can be operably incorporated in a carrier or delivery vector. Useful delivery vectors include but are not limited to biodegradable microcapsules, immuno-stimulating complexes (ISCOMs) or liposomes, and genetically engineered attenuated live carriers such as viruses or bacteria.

Optionally, the DNAs also can be provided with reagents that improve the uptake of the genetic material by cells. For example, the DNA can be formulated with or administered in conjunction with an uptake facilitator reagent selected from the group consisting of benzoic acid esters, anilides, amidines, and urethans.

### B. Antigens

In other embodiments, the pharmaceutically acceptable composition comprises a plurality of developmental cell antigens. The plurality of developmental cell antigens can be in any form including, for example, whole cell lysates, protein or peptide extracts, cellular material (e.g., live or inactivated cells), particulate matter such as, but not limited to, cell debris, apoptotic cells, lipid aggregates such as liposomes, membranous vesicles, microspheres, and the like.

In one embodiment, the plurality of developmental cell antigens comprises developmental cells. The developmental cell can be combined directly with other components that may be present in the pharmaceutically acceptable composition. If desired, the developmental cells can be inactivated to minimize or prevent proliferation once administered to the subject. Any physical, chemical, or biological means of inactivation can be used, including but not limited to irradiation and treatment with mitomycin-C. Developmental cells also can be fixed with chemicals such as glutaraldehyde, paraformaldehyde, or formalin. They also can be in an ionic or non-ionic detergent, such as deoxycholate or octyl glucoside, or treated, for example, using a virus.

If desired, solubilized cell suspensions of developmental cells can be clarified or subjected to any of a number of standard biochemical separation procedures to enrich the plurality of antigens, for example using immunoprecipitation or affinity-purification schemes. The degree of enrichment of the plurality of antigens can be at least 2, 3, 4, 6, 8, 10-fold or more, preferably 100-fold over that of a whole-cell lysate. In one embodiment, antigens associated with the outer membrane of the developmental cell are enriched.

Prior to combining with other components of the pharmaceutically acceptable composition, the developmental cells can be depleted of the chemicals used to treat them, for example by centrifuging and washing fixed cells, or dialysis of the solubilized suspension. Preferably, growth media and/or factors that may be used to culture cells is at least partially removed from the cell preparation. For example, serum (e.g., fetal calf serum, bovine serum) components, or other biological supplements in the culture medium can be removed so as to avoid an immunological side reaction against such components.

For example, the cell components of the pharmaceutically acceptable composition can be washed, such as by repeated centrifugation, into a suitable pharmacologically compatible excipient. Compatible excipients include isotonic saline, with or without a physiologically compatible buffer like phosphate or HEPES and nutrients such as dextrose, physiologically compatible ions, or amino acids, and various culture media suitable for use with the developmental cell populations, particularly those devoid of other immunogenic components. Carrying reagents, such as albumin and blood plasma fractions and nonactive thickening agents, can also be used. Non-active biological components, to the extent that they are present in the pharmaceutically acceptable composition, are preferably derived from the same species, and are even more preferably obtained previously from the subject to be administered.

### C. Dendritic Cell Therapy

One of ordinary skill in the art will recognize that the capacity to generate dendritic cells (DCs) in vitro also can be used in accordance with the present invention for ex vivo loading of DCs with the plurality of developmental cell antigens, or nucleic acids encoding the plurality of developmental cell antigens, and administration of ‘DC vaccines’ as a strategy for eliciting an immune response to the at least one antigen expressed by a cancer cell. Preclinical studies have shown DCs to be potent activators of de novo and recall responses in B and T lymphocytes.

In some embodiments, the present invention provides a method for eliciting in a subject an immune response to the at least one antigen expressed by the cancer cell, the method comprising administering to the subject a pharmaceutically acceptable composition comprising dendritic cells loaded ex vivo with the plurality of developmental cell antigens, or the nucleic acids encoding the plurality of developmental cell antigens, wherein the pharmaceutically acceptable composition, when administered to the subject, elicits the immune response to the at least one antigen.

Accordingly, the developmental cell antigen-primed antigen-presenting cells of the present invention and the developmental antigen-specific T lymphocytes generated with these antigen-presenting cells can be used as active compounds in immunomodulating compositions for prophylactic or therapeutic applications. As described below, in some embodiments, the antigen-primed antigen-presenting cells of the invention can be used for generating cytotoxic T lymphocytes (CTL) (e.g., CD8+ or CD4+ CTL) for adoptive transfer to the subject.

### D. Composition

In other aspects, the present invention provides a pharmaceutically acceptable composition comprising a plurality of antigens expressed by a developmental cell, or nucleic acids encoding the plurality of antigens. The pharmaceutically acceptable composition, when administered to a subject, can elicit an immune response against at least one antigen of a cancer cell. The pharmaceutically acceptable compositions of the present invention can be useful as vaccine compositions for prophylactic or therapeutic treatment of a neoplastic disease or symptoms thereof, particularly for preventing or treating cancer (e.g., a tumor) in the subject.

The plurality of antigens expressed by the developmental cell, or nucleic acids encoding the plurality of antigens, are as described above. In some embodiments, the pharmaceutically acceptable composition further comprises a physiologically acceptable carrier, diluent, or excipient. Techniques for formulating and administering also can be found in Remington's Pharmaceutical Sciences, Mack Publishing Co., Easton, Pa., latest edition.

Pharmaceutically acceptable carriers known in the art include, but are not limited to, sterile water, saline, glucose, dextrose, or buffered solutions. Agents such as diluents, stabilizers (e.g., sugars and amino acids), preservatives, wetting agents, emulsifying agents, pH buffering agents, additives that enhance viscosity, and the like. Preferably, the medium or carrier will produce minimal or no adverse effects.

In other embodiments, the pharmaceutically acceptable composition further comprises a physiologically acceptable adjuvant. Preferably, the adjuvant employed provides for increased immunogenicity. The adjuvant can be one that provides for slow release of antigen (e.g., the adjuvant can be a liposome), or it can be an adjuvant that is immunogenic in its own right thereby functioning synergistically with antigens. For example, the adjuvant can be a known adjuvant or other substance that promotes nucleic acid uptake, recruits immune system cells to the site of administration, or facilitates the immune activation of responding lymphoid cells. Adjuvants include, but are not limited to, immunomodulatory molecules (e.g., cytokines), oil and water emulsions, aluminum hydroxide, glucan, dextran sulfate, iron oxide, sodium alginate, Bacto-Adjuvant, synthetic polymers such as poly amino acids and co-polymers of amino acids, saponin, paraffin oil, and muramyl dipeptide.

In some embodiments, the adjuvant comprises incomplete Freund's adjuvant (Montanide ISA 51) or Corynebacterium granulosum P40.

In one embodiment, the adjuvant comprises an immunomodulatory molecule. For example, the immunomodulatory molecule can be a recombinant protein cytokine, chemokine, or immunostimulatory agent or nucleic acid encoding cytokines, chemokines, or immunostimulatory agents designed to enhance the immunologic response. The adjuvant can be combined or conjugated with the plurality of antigens, or nucleic acids encoding the plurality of antigens. Adjuvants that cannot be expressed from a vector can be administered simultaneously or sequentially, in any order.

Examples of immunomodulatory cytokines include interferons (e.g., IFNα, IFNβ and IFNγ), interleukins (e.g., IL-1, IL-2, IL-3, IL-4, IL-5, IL-6, IL-7, IL-8, IL-9, IL-10, IL-12 and IL-20), tumor necrosis factors (e.g., TNFα and TNFβ), erythropoietin (EPO), FLT-3 ligand, gIp10, TCA-3, MCP-1, MIF, MIP-1α, MIP-1β, Rantes, macrophage colony stimulating factor (M-CSF), granulocyte colony stimulating factor (G-CSF), and granulocyte-macrophage colony stimulating factor (GM-CSF), as well as functional fragments of any of the foregoing. Any immunomodulatory chemokine that binds to a chemokine receptor, i.e., a CXC, CC, C, or CX3C chemokine receptor, also can be used in the context of the present invention. Examples of chemokines include, but are not limited to, Mip1α, Mip-1β, Mip-3α (Larc), Mip-3β, Rantes, Hcc-1, Mpif-1, Mpif-2, Mcp-1, Mcp-2, Mcp-3, Mcp-4, Mcp-5, Eotaxin, Tarc, Elc, I309, IL-8, Gcp-2 Gro-α, Gro-β, Gro-γ, Nap-2, Ena-78, Gcp-2, Ip-10, Mig, I-Tac, Sdf-1, and Bca-1 (Blc), as well as functional fragments of any of the foregoing.

In another embodiment, the adjuvant comprises a cytokine selected from the group consisting of: GM-CSF, G-CSF, IL-2, IL-4, IL-7, IL-12, IL-15, IL-21, TNF-α, and M-CSF. In some embodiments, the adjuvant comprises Freund's adjuvant (Montanide ISA 51) or Corynebacterium granulosum P40. For example, in one embodiment, a sequence encoding GM-CSF or IL-7 can be included on a vector that has an open reading frame encoding a developmental cell antigen or on a separate vector that is administered at or around the same time as the antigen is administered. In another embodiment, the pharmaceutically acceptable composition comprises the plurality of antigens expressed by the developmental cells in the form of developmental cells or cell lines, wherein the developmental cells or cell lines are genetically altered so as to produce a cytokine to provide immunostimulation to generate a specific immune response against the plurality of antigens. In some embodiments, the pharmaceutically acceptable composition comprises the plurality of antigens expressed by the developmental cells in the form of developmental cells or cell lines; and one or more cytokine expressing cell lines. Such compositions can be tailored for each type of cancer or for each subject by including a suitable number of cytokine-producing cells, or with a cocktail of such cells producing a plurality of cytokines at a suitable ratio.

One of ordinary skill in the art knows that methods and compositions of the present invention can be used as part of combination therapies, for example as methods and/or compositions comprising one or more other agents such as, but not limited to, chemotherapeutic, immunotherapeutic, immunomodulatory, anti-angiogenic, and hormonal agents. In various embodiments, the one or more other agents can be a chemotherapeutic agent, naturally occurring or synthetic, for example as described in “Cancer Chemotherapeutic Agents”, American Chemical Society, 1995, W. O. Foye Ed.

In one embodiment, the chemotherapeutic agent is selected from the group consisting of a small molecule receptor antagonists such as vatalanib, SU 11248 or AZD-6474, EGFR or HER2 antagonists such as gefitinib, erlotinib, CI-1033 or Herceptin, antibodies such as bevacizumab, cetuximab, rituximab, DNA alkylating drugs such as cisplatin, oxaliplatin or carboplatin, anthracyclines such as doxorubicin or epirubicin, an antimetabolite such as 5-FU, pemetrexed, gemcitabine or capecitabine, a camptothecin such as irinotecan or topotecan, an anti-cancer drug such as paclitaxel or docetaxel, an epipodophyllotoxin such as etoposide or teniposide, a proteasome inhibitor such as bortezomib or antiinflammatory drugs such as celecoxib or rofecoxib, optionally in form of the pharmaceutically acceptable salts, in form of the hydrates and/or solvates and optionally in the form of the individual optical isomers, mixtures of the individual enantiomers or racemates thereof.

In another embodiment, the chemotherapeutic agent is selected from the group consisting of a small molecule VEGF receptor antagonist such as vatalanib (PTK-787/ZK222584), SU-5416, SU-6668, SU-11248, SU-14813, AZD-6474, AZD-2171, CP-547632, CEP-7055, AG-013736, IM-842 or GW-786034, a dual EGFR/HER2 antagonist such as gefitinib, erlotinib, CI-1033 or GW-2016, an EGFR antagonist such as iressa (ZD-1839), tarceva (OSI-774), PKI-166, EKB-569, HKI-272 or herceptin, an antagonist of the mitogen-activated protein kinase such as BAY-43-9006 or BAY-57-9006, a quinazoline derivative such as 4-[(3-chloro-4-fluorophenyl)amino]-6-{[4-(N,N-dimethylamino)-1-oxo-2-bute-n-1-yl]amino}-7-((S)-tetrahydrofuran-3-yloxy)quinazoline or 4-[(3-chloro-4-fluorophenyl)amino]-6-{[4-(homomorpholin-4-yl)-1-oxo-2-bu-ten-1-yl]amino}-7-[(S)-(tetrahydrofuran-3-yl)oxy]-quinazoline, or a pharmaceutically acceptable salt thereof, a protein kinase receptor antagonist which is not classified under the synthetic small molecules such as atrasentan, rituximab, cetuximab, Avastin™ (bevacizumab), IMC-1C11, erbitux (C-225), DC-101, EMD-72000, vitaxin, imatinib, a protein tyrosine kinase inhibitor which is a fusion protein such as VEGFtrap, an alkylating agent or a platinum compound such as melphalan, cyclophosphamide, an oxazaphosphorine, cisplatin, carboplatin, oxaliplatin, satraplatin, tetraplatin, iproplatin, mitomycin, streptozocin, carmustine (BCNU), lomustine (CCNU), busulfan, ifosfamide, streptozocin, thiotepa, chlorambucil, a nitrogen mustard such as mechlorethamine, an ethyleneimine compound, an alkylsulphonate, daunorubicin, doxorubicin (adriamycin), liposomal doxorubicin (doxil), epirubicin, idarubicin, mitoxantrone, amsacrine, dactinomycin, distamycin or a derivative thereof, netropsin, pibenzimol, mitomycin, CC-1065, a duocarmycin, mithramycin, chromomycin, olivomycin, a phtalanilide such as propamidine or stilbamidine, an anthramycin, an aziridine, a nitrosourea or a derivative thereof, a pyrimidine or purine analogue or antagonist or an inhibitor of the nucleoside diphosphate reductase such as cytarabine, 5-fluorouracile (5-FU), pemetrexed, tegafur/uracil, uracil mustard, fludarabine, gemcitabine, capecitabine, mercaptopurine, cladribine, thioguanine, methotrexate, pentostatin, hydroxyurea, or folic acid, a phleomycin, a bleomycin or a derivative or salt thereof, CHPP, BZPP, MTPP, BAPP, liblomycin, an acridine or a derivative thereof, a rifamycin, an actinomycin, adramycin, a camptothecin such as irinotecan (camptosar) or topotecan, an amsacrine or analogue thereof, a tricyclic carboxamide, an histonedeacetylase inhibitor such as SAHA, MD-275, trichostatin A, CBHA, LAQ824, or valproic acid, an anti-cancer drug from plants such as paclitaxel (taxol), docetaxel or taxotere, a vinca alkaloid such as navelbine, vinblastin, vincristin, vindesine or vinorelbine, a tropolone alkaloid such as colchicine or a derivative thereof, a macrolide such as maytansine, an ansamitocin or rhizoxin, an antimitotic peptide such as phomopsin or dolastatin, an epipodophyllotoxin or a derivative of podophyllotoxin such as etoposide or teniposide, a steganacin, an antimitotic carbamate derivative such as combretastatin or amphetinile, procarbazine, a proteasome inhibitor such as bortezomib, an enzyme such as asparaginase, pegylated asparaginase (pegaspargase) or a thymidine-phosphorylase inhibitor, a gestagen or an estrogen such as estramustine (T-66) or megestrol, an anti-androgen such as flutamide, casodex, anandron or cyproterone acetate, an aromatase inhibitor such as aminogluthetimide, anastrozole, formestan or letrozole, a GNrH analogue such as leuprorelin, buserelin, goserelin or triptorelin, an anti-estrogen such as tamoxifen or its citrate salt, droloxifene, trioxifene, raloxifene or zindoxifene, a derivative of 17β-estradiol such as ICI 164,384 or ICI 182,780, aminoglutethimide, formestane, fadrozole, finasteride, ketoconazole, a LH-RH antagonist such as leuprolide, a steroid such as prednisone, prednisolone, methylprednisolone, dexamethasone, budenoside, fluocortolone or triamcinolone, an interferon such as interferon (3, an interleukin such as IL-10 or IL-12, an anti-TNFα antibody such as etanercept, an immunomodulatory drug such as thalidomide, its R- and S-enantiomers and its derivatives, or revimid (CC-5013), a leukotrien antagonist, mitomycin C, an aziridoquinone such as BMY-42355, AZQ or EO-9, a 2-nitroimidazole such as misonidazole, NLP-1 or NLA-1, a nitroacridine, a nitroquinoline, a nitropyrazoloacridine, a “dual-function” nitro aromatic such as RSU-1069 or RB-6145, CB-1954, a N-oxide of nitrogen mustard such as nitromin, a metal complex of a nitrogen mustard, an anti-CD3 or anti-CD25 antibody, a tolerance induction agent, a biphosphonate or derivative thereof such as minodronic acid or its derivatives (YM-529, Ono-5920, YH-529), zoledronic acid monohydrate, ibandronate sodium hydrate or clodronate disodium, a nitroimidazole such as metronidazole, misonidazole, benznidazole or nimorazole, a nitroaryl compound such as RSU-1069, a nitroxyl or N-oxide such as SR-4233, an halogenated pyrimidine analogue such as bromodeoxyuridine, iododeoxyuridine, a thiophosphate such as WR-272 1, a photo-chemically activated drug such as porfimer, photofrin, a benzoporphyrin derivative, a pheophorbide derivative, merocyanin 540 (MC-540) or tin etioporpurin, an ant-template or an anti-sense RNA or DNA such as oblimersen, a non-steroidal inflammatory drug such as acetylsalicyclic acid, mesalazin, ibuprofen, naproxen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen, indomethacin, sulindac, tolmetin, zomepirac, nabumetone, diclofenac, fenclofenac, alclofenac, bromfenac, ibufenac, aceclofenac, acemetacin, fentiazac, clidanac, etodolac, oxpinac, mefenamic acid, meclofenamic acid, flufenamic acid, nifluminic acid, tolfenamic acid, diflunisal, flufenisal, piroxicam, tenoxicam, lomoxicam, nimesulide, meloxicam, celecoxib, rofecoxib, or a pharmaceutically acceptable salt of a non-steroidal inflammatory drug, a cytotoxic antibiotic, an antibody targeting the surface molecules of cancer cells such as apolizumab or 1D09C3, an inhibitor of metalloproteinases such as TIMP-1 or TIMP-2, Zinc, an inhibitor of oncogenes such as P53 and Rb, a complex of rare earth elements such as the heterocyclic complexes of lanthanides, a photo-chemotherapeutic agent such as PUVA, an inhibitor of the transcription factor complex ESX/DRIP130/Sur-2, an inhibitor of HER-2 expression, such as the heat shock protein HSP90 modulator geldanamycin and its derivative 17-allylaminogeldanamycin or 17-AAG, or a therapeutic agent selected from IM-842, tetrathiomolybdate, squalamine, combrestatin A4, TNP-470, marimastat, neovastat, bicalutamide, abarelix, oregovomab, mitumomab, TLK-286, alemtuzumab, ibritumomab, temozolomide, denileukin diftitox, aldesleukin, dacarbazine, floxuridine, plicamycin, mitotane, pipobroman, plicamycin, tamoxifen and testolactone. Preferred compounds include small molecule VEGF receptor antagonist such as vatalanib (PTK-787/ZK222584), SU-5416, SU-6668, SU-11248, SU-14813, AZD-6474, EGFR/HER2 antagonists such as CI-1033 or GW-2016, an EGFR antagonist such as iressa (gefitinib, ZD-1839), tarceva (erlotinib, OSI-774), PKI-166, EKB-569, HKI-272 or herceptin, an antagonist of the mitogen-activated protein kinase such as BAY-43-9006 or BAY-57-9006, atrasentan, rituximab, cetuximab, Avastin™ (bevacizumab), IMC-1C11, erbitux (C-225), DC-101, EMD-72000, vitaxin, imatinib, an alkylating agent or a platinum compound such as melphalan, cyclophosphamide, cisplatin, carboplatin, oxaliplatin, satraplatin, daunorubicin, doxorubicin (adriamycin), liposomal doxorubicin (doxil), epirubicin, idarubicin, a pyrimidine or purine analogue or antagonist or an inhibitor of the nucleoside diphosphate reductase such as cytarabine, 5-fluorouracile (5-FU), pemetrexed, tegafur/uracil, gemcitabine, capecitabine, mercaptopurine, methotrexate, an anti-cancer drug such as paclitaxel (taxol) or docetaxel, a vinca alkaloid such as navelbine, vinblastin, vincristin, vindesine or vinorelbine, an antimitotic peptide such as dolastatin, an epipodophyllotoxin or a derivative of podophyllotoxin such as etoposide or teniposide, a non-steroidal inflammatory drug such as meloxicam, celecoxib, rofecoxib, an antibody targeting the surface molecules of cancer cells such as apolizumab or ID09C3 or the heat shock protein HSP90 modulator geldanamycin and its derivative 17-allylaminogeldanamycin or 17-AAG.

In another embodiment, the chemotherapeutic agent is selected from the group consisting of compounds interacting with or binding tubulin, synthetic small molecule VEGF receptor antagonists, small molecule growth factor receptor antagonists, inhibitors of the EGF receptor and/or VEGF receptor and/or integrin receptors or any other protein tyrosine kinase receptors which are not classified under the synthetic small-molecules, inhibitors directed to EGF receptor and/or VEGF receptor and/or integrin receptors or any other protein tyrosine kinase receptors, which are fusion proteins, compounds which interact with nucleic acids and which are classified as alkylating agents or platinum compounds, compounds which interact with nucleic acids and which are classified as anthracyclines, as DNA intercalators or as DNA cross-linking agents, including DNA minor-groove binding compounds, anti-metabolites, naturally occurring, semi-synthetic or synthetic bleomycin type antibiotics, inhibitors of DNA transcribing enzymes, and especially the topoisomerase I or topoisomerase II inhibitors, chromatin modifying agents, mitosis inhibitors, anti-mitotic agents, cell-cycle inhibitors, proteasome inhibitors, enzymes, hormones, hormone antagonists, hormone inhibitors, inhibitors of steroid biosynthesis, steroids, cytokines, hypoxia-selective cytotoxins, inhibitors of cytokines, lymphokines, antibodies directed against cytokines, oral and parenteral tolerance induction agents, supportive agents, chemical radiation sensitizers and protectors, photo-chemically activated drugs, synthetic poly- or oligonucleotides, optionally modified or conjugated, non-steroidal anti-inflammatory drugs, cytotoxic antibiotics, antibodies targeting the surface molecules of cancer cells, antibodies targeting growth factors or their receptors, inhibitors of metalloproteinases, metals, inhibitors of oncogenes, inhibitors of gene transcription or of RNA translation or protein expression, complexes of rare earth elements, and photo-chemotherapeutic agents.

In other embodiments, the chemotherapeutic agent is selected from the group consisting of paclitaxel (taxol), docetaxel, a vinca alkaloid such as navelbine, vinblastin, vincristin, vindesine or vinorelbine, an alkylating agent or a platinum compound such as melphalan, cyclophosphamide, an oxazaphosphorine, cisplatin, carboplatin, oxaliplatin, satraplatin, tetraplatin, iproplatin, mitomycin, streptozocin, carmustine (BCNU), lomustine (CCNU), busulfan, ifosfamide, streptozocin, thiotepa, chlorambucil, a nitrogen mustard such as mechlorethamine, an immunomodulatory drug such as thalidomide, its R- and S-enantiomers and its derivatives, or revimid (CC-5013)), an ethyleneimine compound, an alkylsulphonate, daunorubicin, doxorubicin (adriamycin), liposomal doxorubicin (doxil), epirubicin, idarubicin, mitoxantrone, amsacrine, dactinomycin, distamycin or a derivative thereof, netropsin, pibenzimol, mitomycin, CC-1065, a duocarmycin, mithramycin, chromomycin, olivomycin, a phtalanilide such as propamidine or stilbamidine, an anthramycin, an aziridine, a nitrosourea or a derivative thereof, a pyrimidine or purine analogue or antagonist or an inhibitor of the nucleoside diphosphate reductase such as cytarabine, 5-fluorouracile (5-FU), uracil mustard, fludarabine, gemcitabine, capecitabine, mercaptopurine, cladribine, thioguanine, methotrexate, pentostatin, hydroxyurea, or folic acid, an acridine or a derivative thereof, a rifamycin, an actinomycin, adramycin, a camptothecin such as irinotecan (camptosar) or topotecan, an amsacrine or analogue thereof, a tricyclic carboxamide, an histonedeacetylase inhibitor such as SAHA, MD-275, trichostatin A, CBHA, LAQ824, or valproic acid, a proteasome inhibitor such as bortezomib, a small molecule VEGF receptor antagonist such as vatalanib (PTK-787/ZK222584), SU-5416, SU-6668, SU-11248, SU-14813, AZD-6474, AZD-2171, CP-547632, CEP-7055, AG-013736, IM-842 or GW-786034, an antagonist of the mitogen-activated protein kinase such as BAY-43-9006 or BAY-57-9006, a dual EGFRMER2 antagonist such as gefitinib, erlotinib, CI-1033 or GW-2016, an EGFR antagonist such as iressa (ZD-1839), tarceva (OSI-774), PKI-166, EKB-569, HKI-272 or herceptin, a quinazoline derivative such as 4-[(3-chloro-4-fluorophenyl)amino]-6-{[-4-(N,N-dimethylamino)-1-oxo-2-but-en-1-yl]amino}-7-((S)-tetrahydrofuran-3-yloxy)-quinazoline or 4-[(3-chloro-4-fluoro-phenyl)amino]-6-{[4-(homomorpholin-4-yl)-1-oxo-2-bu-ten-1-yl]amino}-7-[(S)-(tetrahydrofuran-3-yl)oxy]-quinazoline, or a pharmaceutically acceptable salt thereof, an inhibitor of the transcription factor complex ESX/DRIP130/Sur-2, an inhibitor of HER-2 expression, such as the heat shock protein HSP90 modulator geldanamycin and its derivative 17-allylaminogeldanamycin or 17-AAG, a protein kinase receptor antagonist which is not classified under the synthetic small molecules such as atrasentan, rituximab, cetuximab, Avastin™ (bevacizumab), IMC-1C11, erbitux (C-225), DC-101, EMD-72000, vitaxin, imatinib, and an antibody targeting the surface molecules of cancer cells such as apolizumab or 1D09C3.

In some other embodiments, the chemotherapeutic agent is a compound which reduces the transport of hyaluronan mediated by one or more ABC transporters, or drug transport inhibitor, such as a P-glycoprotein (P-gp) inhibitor molecule or inhibitor peptide, an MRP1 inhibitor, an antibody directed against and capable of blocking the ABC transporter, an antisense oligomer, iRNA, siRNA or aptamer directed against one or more ABC transporters. Examples of P-glycoprotein (P-gp) inhibitor molecules in accordance with the present invention are zosuquidar (LY 335973), its salts (especially the trichloride salt) and its polymorphs, cyclosporin A (also known as cyclosporine), verapamil or its R-isomer, tamoxifen, quinidine, d-alpha tocopheryl polyethylene glycol 1000 succinate, VX-710, PSC833, phenothiazine, GF120918 (II), SDZ PSC 833, TMBY, MS-073, S-9788, SDZ 280-446, XR(9051) and functional derivatives, analogues and isomers of these.

In one embodiment, the present invention relates to the use of a pharmaceutical composition as defined herein for the preparation of a medicament for the treatment of cancer.

### E. Adoptive Immunotherapy

In various other aspects, the plurality of developmental cell antigens or nucleic acids encoding the plurality of developmental cell antigens also can provide for compositions and methods for providing cancer antigen-primed antigen-presenting cells, and cancer antigen-specific T lymphocytes generated with these antigen-presenting cells, e.g., for use as active compounds in immunomodulating compositions and methods for prophylactic or therapeutic applications for cancer.

Accordingly, in one aspect, the invention provides a method for making cancer antigen-primed, antigen-presenting cells by:

contacting antigen-presenting cells with a plurality of developmental cell antigens, or nucleic acids encoding the plurality of developmental cell antigens, in vitro under a condition sufficient for the plurality of developmental cell antigens to be presented by the antigen-presenting cells.

The plurality of developmental cell antigens, or nucleic acids encoding the plurality of developmental cell antigens, are as described above.

The plurality of developmental cell antigens, or nucleic acids encoding the plurality of developmental cell antigens, can be contacted with a homogenous, substantially homogenous, or heterogeneous composition comprising antigen-presenting cells. For example, the composition can include but is not limited to whole blood, fresh blood, or fractions thereof such as, but not limited to, peripheral blood mononuclear cells, buffy coat fractions of whole blood, packed red cells, irradiated blood, dendritic cells, monocytes, macrophages, neutrophils, lymphocytes, natural killer cells, and natural killer T cells. If, optionally, precursors of antigen-presenting cells are used, the precursors can be cultured under suitable culture conditions sufficient to differentiate the precursors into antigen-presenting cells. Preferably, the antigen-presenting cells (or, optionally, precursors) are selected from monocytes, macrophages, cells of myeloid lineage, B cells, dendritic cells, or Langerhans cells.

The amount of the plurality of developmental cell antigens, or nucleic acids encoding the plurality of developmental cell antigens, to be placed in contact with antigen-presenting cells can be determined by one of ordinary skill in the art by routine experimentation. Generally, antigen-presenting cells are contacted with the plurality of developmental cell antigens, or nucleic acids encoding the plurality of developmental cell antigens, for a period of time sufficient for cells to present the processed forms of the antigens for the modulation of T cells. In one embodiment, antigen-presenting cells are incubated in the presence of the plurality of developmental cell antigens, or nucleic acids encoding the plurality of developmental cell antigens, for less than about a week, illustratively, for about 1 minute to about 48 hours, about 2 minutes to about 36 hours, about 3 minutes to about 24 hours, about 4 minutes to about 12 hours, about 6 minutes to about 8 hours, about 8 minutes to about 6 hours, about 10 minutes to about 5 hours, about 15 minutes to about 4 hours, about 20 minutes to about 3 hours, about 30 minutes to about 2 hours, and about 40 minutes to about 1 hour. The time and amount of antigens, or nucleic acids encoding the antigens, necessary for the antigen presenting cells to process and present the antigens can be determined, for example using pulse-chase methods wherein contact is followed by a washout period and exposure to a read-out system e.g., antigen reactive T cells.

Typically, the length of time necessary for an antigen-presenting cell to present an antigen on its surface can vary depending on a number of factors including the antigen or form (e.g., peptide versus encoding polynucleotide) of antigen employed, its dose, and the antigen-presenting cell employed, as well as the conditions under which antigen loading is undertaken. These parameters can be determined by the skilled artisan using routine procedures. Efficiency of priming of an antigen-presenting cell can be determined by assaying T cell cytotoxic activity in vitro or using antigen-presenting cells as targets of CTLs. Other methods that can detect the presence of antigen on the surface of antigen-presenting cells are also contemplated by the presented invention.

A number of methods for delivery of antigens to the endogenous processing pathway of antigen-presenting cells are known. Such methods include but are not limited to methods involving pH-sensitive liposomes, coupling of antigens to potent adjuvants, apoptotic cell delivery, pulsing cells onto dendritic cells, delivering recombinant chimeric virus-like particles (VLPs) comprising antigen to the MHC class I processing pathway of a dendritic cell line.

In one embodiment, solubilized developmental cell antigens are incubated with antigen-presenting cells. In other embodiments, developmental cell antigens can be coupled to a cytolysin to enhance the transfer of the antigens into the cytosol of an antigen-presenting cell for delivery to the MHC class I pathway. Exemplary cytolysins include saponin compounds such as saponin-containing Immune Stimulating Complexes (ISCOMs), pore-forming toxins (e.g., an alpha-toxin), and natural cytolysins of gram-positive bacteria such as listeriolysin O (LLO), streptolysin O (SLO), and perfringolysin O (PFO).

By way of another example, in other embodiments, antigen-presenting cells, preferably dendritic cells and macrophage, can be isolated according to methods known in the art and transfected with polynucleotides by methods known in the art for introducing nucleic acids encoding the plurality of antigens into the APCs. Transfection reagents and methods (e.g., SuperFect®) also are commercially available. For example, RNAs encoding the plurality of antigens can be provided in a suitable medium (e.g., Opti-MEM®) and combined with a lipid (e.g., a cationic lipid) prior to contact with APCs. Non-limiting examples of lipids include LIPOFECTIN™, LIPOFECTAMINE™, DODAC/DOPE, and CHOL/DOPE. The resulting polynucleotide-lipid complex can then be contacted with APCs. Alternatively, the polynucleotide can be introduced into APCs using techniques such as electroporation or calcium phosphate transfection. The polynucleotide-loaded APCs can then be used to stimulate cytotoxic T lymphocyte (CTL) proliferation in vivo or ex vivo. In one embodiment, the ex vivo expanded CTL is administered to the subject in a method of adoptive immunotherapy. The ability of the polynucleotide-loaded antigen-presenting cells to stimulate a CTL response can be determined by known methods, for example by assaying the ability of effector cells to lyse a target cell. Methods and compositions using antigen-presenting cells loaded with e.g., RNA are described in U.S. Pat. No. 6,306,388 to Nair et al., which is incorporated herein by reference for its teaching of methods of generation and use of APCs loaded with RNA.

In another aspect, the present invention provides a composition comprising antigen-presenting cells that have been contacted in vitro with a plurality of developmental cell antigens, or nucleic acids encoding the plurality of developmental cell antigens, under a condition sufficient for the plurality of developmental cell antigens to be presented by the antigen-presenting cells.

In another aspect, the present invention provides a method for preparing lymphocytes specific for the at least one antigen of a cancer cell. The method comprises contacting lymphocytes with the antigen-presenting cells described above under conditions sufficient to produce at least one cancer antigen-specific lymphocyte capable of eliciting an immune response against a cancer cell. Thus, the antigen-presenting cells also can be used to provide lymphocytes, including T lymphocytes and B lymphocytes, for eliciting an immune response against the at least one antigen of the cancer cell.

In one embodiment, a preparation of T lymphocytes is contacted with the antigen-presenting cells described above for a period of time, preferably for at least about 24 hours, for priming the T lymphocytes to the developmental cell antigens presented by the antigen-presenting cells.

For example, in another embodiment, a population of antigen-presenting cells can be co-cultured with a heterogeneous population of peripheral blood T lymphocytes together with a plurality of developmental cell antigens or nucleic acids comprising the plurality of antigens. The cells can be co-cultured for a period of time and under conditions sufficient for the plurality of antigens or their processed forms to be presented by the antigen-presenting cells and the antigen-presenting cells to prime a population of T lymphocytes to respond to the at least one antigen of the cancer cell. Accordingly, T lymphocytes and B lymphocytes that are primed to respond to the at least one antigen of the cancer cell can be prepared.

As described herein, the ability to induce lymphocytes to exhibit an immune response can be determined by any method including, but not limited to, determining T lymphocyte cytolytic activity in vitro using for example developmental cell antigen-specific antigen-presenting cells as targets of developmental cell antigen-specific cytolytic T lymphocytes (CTL); assaying developmental cell antigen-specific T lymphocyte proliferation; and determining B cell response to developmental cell antigen using, for example, ELISA methods.

T lymphocytes can be obtained from any suitable source such as peripheral blood, spleen, and lymph nodes. The T lymphocytes can be used as crude preparations or as partially purified or substantially purified preparations, which can be obtained by standard techniques including, but not limited to, methods involving immunomagnetic or flow cytometry techniques using antibodies.

Also contemplated within the scope of the present invention are cells that have been modified genetically (e.g., T-cells genetically engineered to express cell-specific antibodies on their surface) for specific recognition of a cell. In some embodiments, antigen-specific T cells are modified by gene transfer techniques known in the art to express one or more heterologous genes, for example a marker gene or a gene whose gene product can enhance or impart a particular phenotype or function to the antigen-specific T cell. Thus, for example, a marker gene can be expressed within activated T cells responding to antigen pulsed dendritic cells and allow for the selective enrichment and modification of antigen-specific T cells. By way of another example, antigen-specific T cells can be modified to express a receptor (e.g., a chemokine receptor) to migrate toward a ligand of the receptor in vitro and in vivo. Antigen-specific T cells that have been modified for selective enrichment or for targeting are described by Mitchell et al., Human Gene Therapy, 19:511 (2008), which is incorporated herein by reference for its teaching of modified T cells. In other aspects, the present invention provides a composition comprising the antigen-presenting cells or the lymphocytes described above, and a pharmaceutically acceptable carrier and/or diluent. In some embodiments, the composition further comprises an adjuvant as described above.

In another aspect, the present invention provides a method for eliciting an immune response to the at least one antigen of the cancer cell, the method comprising administering to the subject the antigen-presenting cells or the lymphocytes described above in effective amounts sufficient to elicit the immune response. In some embodiments, the invention provides a method for treatment or prophylaxis of a neoplastic disease or symptoms associated therewith, the method comprising administering to the subject an effective amount of the antigen-presenting cells or the lymphocytes described above. In one embodiment, the antigen-presenting cells or the lymphocytes are administered systemically, preferably by injection. Alternately, one can administer locally rather than systemically, for example, via injection directly into tissue, preferably in a depot or sustained release formulation. Furthermore, one can administer in a targeted drug delivery system, for example, in a liposome that is coated with tissue-specific antibody. The liposomes can be targeted to and taken up selectively by the tissue. In another embodiment, the invention provides use of the antigen-presenting cells or the lymphocytes in the preparation of a medicament for eliciting an immune response to the at least one antigen of the cancer cell, preferably for treating or preventing cancer.

Accordingly, the antigen-primed antigen-presenting cells of the present invention and the antigen-specific T lymphocytes generated with these antigen-presenting cells can be used as active compounds in immunomodulating compositions for prophylactic or therapeutic applications for cancer. In some embodiments, the developmental cell antigen-primed antigen-presenting cells of the invention can be used for generating CD8+, CD4+ CTL, or B-cell lymphocytes for adoptive transfer to the subject. Thus, for example, developmental cell antigen-specific CTLs can be adoptively transferred for therapeutic purposes in subjects afflicted with a malignant tumor such as a glioma.

The developmental cell antigen-presenting cells and the lymphocytes described above can be administered to a subject, either by themselves or in combination, for eliciting an immune response, particularly for eliciting an immune response to the at least one antigen of the cancer cell. Such cell-based compositions are useful, therefore, for treating or preventing cancer. The cells can be introduced into a subject by any mode that elicits the desired immune response to the at least one antigen of the cancer cell. Furthermore, the cells can be derived from the subject (i.e., autologous cells) or from a different subject that is MHC matched or mismatched with the subject (e.g., allogeneic). The injection site can be selected from subcutaneous, intraperitoneal, intramuscular, intradermal, intravenous, or intralymphoid.

Single or multiple administrations of the cells can be carried out with cell numbers and treatment being selected by the care provider (e.g., physician). Preferably, the cells are administered in a pharmaceutically acceptable carrier. Suitable carriers can be the growth medium in which the cells were grown, or any suitable buffering medium such as phosphate buffered saline. The cells can be administered alone or as an adjunct therapy in conjunction with other therapeutics.

Accordingly, the invention contemplates methods for treatment and/or prophylaxis of cancer, the method comprising administering to a subject in need of such treatment or prophylaxis a therapeutically/prophylactically effective amount of a composition as described herein.

Techniques for formulating and administering can be found in Remington's Pharmaceutical Sciences, Mack Publishing Co., Easton, Pa., latest edition. Suitable routes can, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. For injection, the therapeutic/prophylactic compositions of the present invention can be formulated in aqueous solutions, preferably in physiologically compatible buffers such as Hanks' solution, Ringer's solution, or physiological saline buffer.

### F. Antibodies

The compositions of the present invention also can be used to raise antibodies against the at least one antigen expressed by a cancer cell. Accordingly, in other aspects, the composition and methods of the present invention provide one or more antibodies against the at least one antigen of the cancer cell, which antibodies themselves have many uses such as, for example, passive immunization or target-specific delivery for effectors as well as uses for diagnostic tests and kits based upon immunological binding. Thus, in some embodiments, the present invention provides cancer cell-specific antibodies that can be used in therapeutic and/or diagnostic applications.

The cancer cell-specific antibodies of the present invention can be used in screening or diagnostic applications. The cancer cell-specific antibodies according to the present invention are valuable for in vitro and in vivo diagnostic purposes. For example, the cancer cell-specific can be used in western blots, immunoprecipitation, enzyme-linked immunosorbent assay (ELISA), fluorescence activated cell sorting (FACS), indirect immunofluoresence microscopy, immunohistochemistry (IHC), etc. In one embodiment, the present invention provides an immunological method for determining a cancer cell, the method comprising contacting the cell with at least one cancer cell-specific antibody as disclosed herein.

For example, the cancer cell-specific antibodies can be used as diagnostic agents for assaying for the detection of cancer expressing cells. The cancer binding antibodies of the present invention should be particularly suitable as diagnostic agents given their binding affinity to cancer. Essentially, a sample suspected of containing cancer expressing cells (e.g., cancer cells) will be incubated with the antibodies for a sufficient time to permit immunological interactions to occur. Those skilled in the art will recognize that there are many variations in these basic procedures. These variations include, for example, RIA, ELISA, precipitation, agglutination, complement fixation and immunofluorescence. Preferably, the subject antibodies will be labeled to permit the detection of antibody-cancer immunocomplexes. Further, the cancer cell-specific antibodies of the present invention are also useful for detection and quantitation of cancer in vitro, to kill and eliminate cancer-expressing cells from a population of mixed cells as a step in the purification of other cells.

The labels that are used in making labeled versions of the antibodies include moieties that may be detected directly, such as radiolabels and fluorochromes, as well as moieties, such as enzymes, that must be reacted or derivatized to be detected. Radiolabels include, but are not limited to, ⁹⁹Tc, ²⁰³Pb, ⁶⁷Ga, ⁶⁸Ga, ⁷²As, ¹¹¹In, ^{113m}In, ⁹⁷Ru, ⁶²Cu, ⁶⁴¹Cu, ⁵²Fe, ^{52m}Mn, ⁵¹Cr, ¹⁸⁶Re, ¹⁸⁸Re, ⁷⁷As, ⁹⁰Y, ⁶⁷Cu, ¹⁶⁹Er, ¹²¹Sn, ¹²⁷Te, ¹⁴²Pr, ¹⁴³Pr, ¹⁹⁸Au, ¹⁹⁹Au, ¹⁶¹Tb, ¹⁰⁹Pd, ¹⁶⁵_{Dy,} ¹⁴⁹Pm, ¹⁵¹Pm, ¹⁵³Sm, ¹⁵⁷Gd, ¹⁵⁹Gd, ¹⁶⁶Ho, ¹⁷²Tm, ¹⁶⁹Yb, ¹⁷⁵Yb, ¹⁷⁷Lu, ¹⁰⁵Rh, and ¹¹¹Ag. The radiolabel can be detected by any of the currently available counting procedures.

An enzyme label can be detected by any of the currently utilized calorimetric, spectrophotometric, fluorospectrophotometric or gasometric techniques. The enzyme is combined with the antibody with bridging molecules such as carbodiimides, periodate, diisocyanates, glutaraldehyde and the like. Many enzymes which can be used in these procedures are known and can be utilized. Examples are perioxidase, alkaline phosphatase, .beta.-glucuronidase, β-D-glucosidase, urease, glucose oxidase plus peroxidase, galactose oxidase plus peroxidase and acid phosphatase. Fluorescent materials which may be used include, for example, fluorescein and its derivatives, rhodamine and its derivatives, auramine, dansyl, umbelliferone, luciferia, 2,3-dihydrophthalazinediones, horseradish peroxidase, alkaline phosphatase, lysozyme, and glucose-6-phosphate dehydrogenase. The antibodies may be tagged with such labels by known methods. For instance, coupling agents such as aldehydes, carbodiimides, dimaleimide, imidates, succinimides, bis-diazotized benzadine and the like may be used to tag the antibodies with the above-described fluorescent, chemiluminescent, and enzyme labels. Various labeling techniques are described in Morrison, Methods in Enzymology, (1974), 32B, 103; Syvanen et al., J. Biol. Chem., (1973), 284, 3762; and Bolton and Hunter, Biochem J., (1973), 133, 529.

The antibodies and labeled antibodies may be used in a variety of immunoimaging or immunoassay procedures to detect the presence of cancer in a patient or monitor the status of such cancer in a patient already diagnosed to have it. When used to monitor the status of a cancer, a quantitative immunoassay procedure must be used. If such monitoring assays are carried out periodically and the results compared, a determination may be made regarding whether the patient's tumor burden has increased or decreased. Common assay techniques that may be used include direct and indirect assays.

For example, in the case of therapeutic applications, the antibodies can be used to inhibit a target involved in disease progression or to bring about the cytotoxic death of target cells. Also, such therapeutic antibodies can inhibit a signaling pathway or induce antibody-dependent cell-mediated cytotoxicty, complement-dependent cytotoxicty, etc.

The cancer cell-specific antibodies of this invention thus provide effective targeting moieties that can, but need not, be transiently or permanently coupled to an effector (thereby forming a hybrid molecule or chimeric moiety) and used to direct that effector to a particular target cell (e.g., a cancer cell).

The effector molecule refers to a molecule or group of molecules that is to be specifically transported to the target cell. The effector molecule typically has a characteristic activity that is to be delivered to the target cell. Effector molecules include, but are not limited to cytotoxins, labels, radionuclides (e.g., ²¹¹At), ligands, antibodies, drugs, liposomes, epitope tags, and the like. Preferred effectors include cytotoxins (e.g., Pseudomonas exotoxin, gelonin, ricin, abrin, Diphtheria toxin, and the like), immunomodulators (e.g., IL2, TNF-α, GM-CSF, B 7.1, H60), or cytotoxic drugs or prodrugs, in which case the hybrid molecule may act as a potent cell-killing agent specifically targeting the cytotoxin to cells bearing the cancer target.

Further examples of effectors include, but are not limited to, granzyme, luciferase, vascular endothelial growth factor, b-lactamase, Tr-apo-1, Ang II, TAT, alkylating agents, daunomycin, adriamycin, chlorambucil, anti-metabolites (e.g., methotrexate), modaccin A chain, alpha-sarcin, Aleurites fordii proteins, dianthin proteins, Phytolacca americana proteins (PAPI, PAPII, and PAP-S), Momordica charantia inhibitor, curcin, crotin, Sapaonaria officinalis inhibitor, mitogellin, restrictocoin, phenomycin and enomycin.

In still other embodiments, the effector can include a liposome encapsulating a drug (e.g., an anti-cancer drug such as doxirubicin, vinblastine, taxol, or other chemotherapeutic agents described herein), an antigen that stimulates recognition of the bound cell by components of the immune system, and antibody that specifically binds immune system components and directs them to the cancer bearing cell, and the like.

Other suitable effector molecules include pharmacological agents or encapsulation systems containing various pharmacological agents. Thus, the targeting molecule of the hybrid molecule may be attached directly to a drug that is to be delivered directly to the tumor. Such drugs are well known to those of skill in the art and include, but are not limited to, doxirubicin, vinblastine, genistein, an antisense molecule, the various other chemotherapeutic agents described herein, and the like.

Alternatively, the effector molecule may be an encapsulation system, such as a viral capsid, a liposome, or micelle that contains a therapeutic composition such as a drug, a nucleic acid (e.g., an antisense nucleic acid), or another therapeutic moiety that is preferably shielded from direct exposure to the circulatory system. Methods of preparing liposomes attached to antibodies are well known to those of skill in the art. See, for example, U.S. Pat. No. 4,957,735; Connor, J. et al. (1985) Pharmacol. Ther., 28: 341-365.

### III. Kits

The compositions of the present invention can be supplied in unit dosage or kit form. Kits can comprise various components of the pharmaceutically acceptable composition or vaccines thereof provided in separate containers as well as various other active ingredients or agents including chemotherapeutic agents. For example, the containers can separately comprise the plurality of antigens expressed by a developmental cell or nucleic acids encoding the plurality of antigens such that when combined with other components of the kit together constitute a pharmaceutically acceptable composition in unit dosage or multiple dosage form. Preferred kits at least comprise, in separate containers, a source of antigens (e.g., the plurality of antigens expressed by a developmental cell or nucleic acids encoding the plurality of antigens); and one or more adjuvants (e.g., cytokines). The kit can further comprise a physiologically acceptable carrier, diluent, or excipient in a separate container. Optionally, the kit can further comprise a delivery agent such as nanoparticles or transfection reagents. Packaged compositions and kits of this invention also can include instructions for storage, preparation, and administering.

The present invention will be illustrated in more detail by way of Examples, but it is to be noted that the invention is not limited to the Examples.

### EXAMPLES

### Example 1

### Vaccination with RNA is Effective in Protection Against Growth of Malignant Astrocytoma

### Tumor Cell Lines and Animal Models

The SMA 560 cell line was derived from an intracerebral transplant of a spontaneous astrocytoma from a VM/Dk mouse (H-2^{b}) and were grown in zinc option medium (GIBCO BRL, Gaithersburg, Md.) containing 5% (vol/vol) fetal calf serum (FCS). All cell lines were shown to be free from Mycoplasma contamination. All experiments used 6-12 wk-old female VM/Dk mice bred and maintained in a virus-free environment in accordance with the Laboratory Animal Resources Commission standards.

### RNA-NP Complexes

Total tumor RNA was isolated from SMA560 tumor cells using standard methods (e.g., Ashley et al., J. Experimental Medicine, 186:1177 (1997). Embryonic RNA from embryonic development day 10 (E10) (Zyagen, San Diego, Calif.) or day 18 (E18) murine embryos (Ambion, Austin, Tex.), embryonic brain total RNA (Zyagen, San Diego, Calif.) were purchased. Complexing of RNA with Superfect® dendrimer nanoparticles (Qiagen, Valencia, Calif.) was performed at room temperature following manufacturer's instructions.

In vivo RNA Vaccination

VM/Dk mice were untreated or immunized once or three times at weekly intervals intradermally at the base of both ears with NP-RNA (75 μg NP:25 μg RNA) complexes in a 100 μL volume (50 μL at base of each ear). One week after the third immunization mice were challenged intracranially with 5000 syngeneic spontaneously-derived astrocytoma cells (SMA50). In some experiments, 1 μg murine GM-CSF (PeproTech, Inc., Rocky Hill, N.J.) was co-injected with NP-RNA complexes by adding 1 μg GM-CSF in 10 μL PBS to NP-RNA complexes just prior to intradermal injection.

### Intracranial Tumor Challenge

Tumor cells were harvested, mixed with an equal volume of 10% methylcellulose in PBS, and loaded into 250-μL Hamilton syringes (Hamilton, Reno, Nev.). Mice (VM/Dk or athymic BALB/c) were anesthetized with a mixture of xylazine/ketamine and placed into a stereotactic frame (Kopf Instruments, Tujunga, Calif.). The injection needle was positioned 2 mm to the right of bregma and 4 mm below the surface of the skull. Cells (1.0×10⁴) in a volume of 5 μL were delivered into the right cerebral hemisphere. After injection, the skull hole was closed with bone wax and the wound was closed with surgical staples (Stoelting Co., Wood Dale, Ill.). Mice were monitored daily and sacrificed when moribund according to Laboratory Animal Resources Commission standards. Survival was analyzed using Kaplan-Meier method.

### Intracranial Tumor Treatment

Tumor cells were harvested and implanted as described above. Treatment with NP-RNA complexes as described above was initiated at Day 4 after tumor implantation and survival of mice monitored daily. Mice were sacrificed when moribund according to Laboratory Animal Resources Commission standards. Survival was analyzed using Kaplan-Meier method.

### Example 2

### Vaccination with Total Embryonic RNA is More Effective than Total Tumor RNA in Treating Established Malignant Gliomas

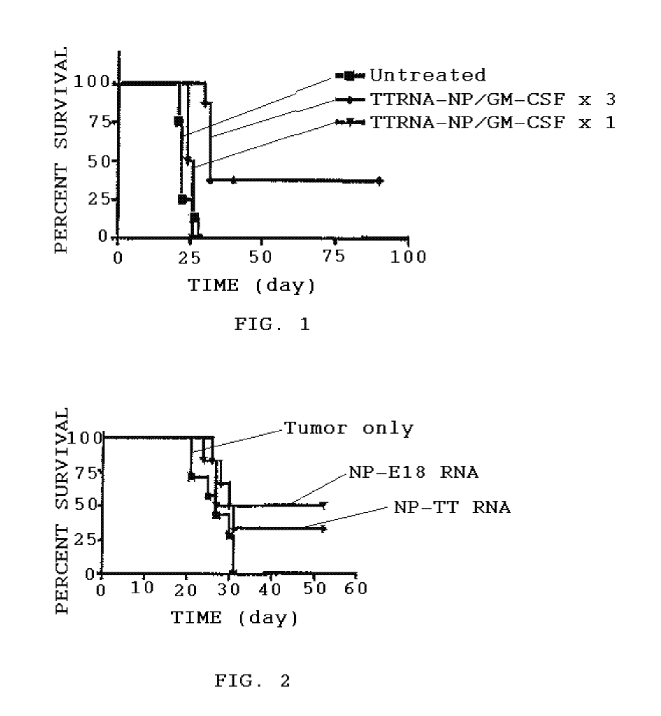

Twenty-five μg of total tumor RNA (TTRNA), and either 25 μg or 75 μg of GM-CSF RNA, was complexed with 75 μg of dendrimer nanoparticles (NP). The RNA:nanoparticles complexes (TTRNA-NP) were delivered to VM/Dk mice by intradermal injection at the base of the ear one week prior to lethal intracranial challenge with the syngeneic astrocytoma line SMA-560. As shown in FIG. 1, vaccination with total tumor RNA and GM-CSF was effective in protection against growth of malignant astrocytoma.

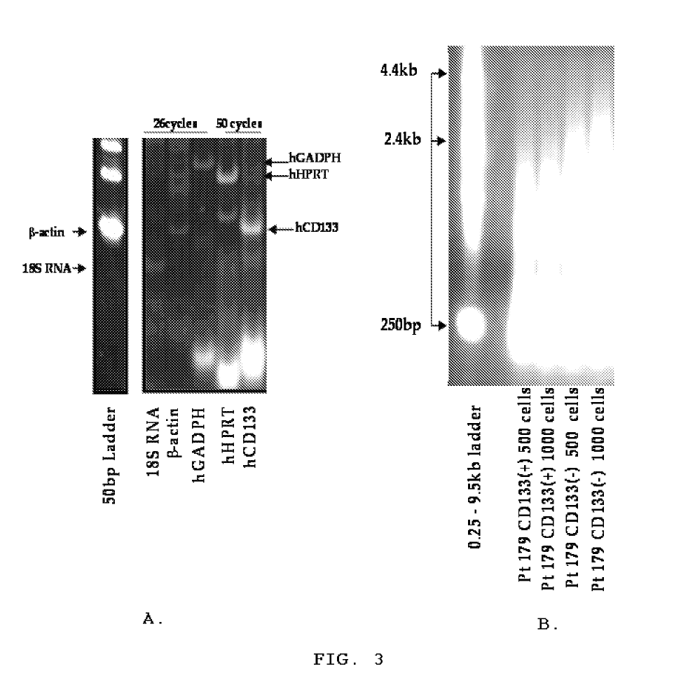

Twenty-five μg of embryonic day 18 (E18) total embryonic RNA (E18 RNA) or total tumor RNA (TTRNA) from SMA-560 astrocytomas was complexed with 75 μg dendrimer nanoparticles (NP). The RNA:nanoparticles complexes were delivered as a single intradermal injection to mice bearing day 4 intracranial astrocytomas. Two vaccinations with total tumor RNA nanoparticles (NP-TTRNA) were given. As shown in FIG. 2, all untreated mice died within 30 days of tumor implantation. 50% of mice vaccinated once with total embryonic RNA (NP-E18 RNA) were cured of established tumor with a single vaccination while two vaccinations of total tumor RNA nanoparticles (NP-TTRNA) cured 33% of mice. The results also indicate that embryonic antigens can be used as an effective source of developmental antigens against malignant glioma. Due to the ready availability and capacity to amplify embryonic tissue RNA, these results also support the rationale for embryonic RNA vaccines as a universal platform for vaccination against brain tumors and the numerous other cancers that share gene expression patterns with developmental cells.

### Example 3

### ex vivo Generation of Autologous DC

Contents of a Leukapheresis Product (LP) bag was placed in a 1 L sterile corning bottle and an equal volume of phosphate buffered saline (PBS) was added to dilute the LP. Using centrifuge tubes, 20 mL of Histopaque™ (Sigma #1077-1) was gently over layered with 30 mL of the diluted LP, then spun at 1300×g for 25 minutes. The interface (1 interface per tube) was removed; PBS was added to 50 mL; and cells pelleted for 5 minutes at 500×g room temperature (RT). The supernatant was decanted and the pellet was resuspended in 50 mL PBS, then pelleted as above. The supernatant was decanted and 2 pellets were combined in 50 mL PBS, then pelleted as above. Again, the supernatant was decanted and 2 pellets were combined in 50 mL PBS, then pelleted as above. Pellets were combined and washed with PBS until all of the cells were combined into 1 tube.

Trypan blue exclusion was performed to determine the number of live and dead cells via with the aid of a hematocytometer. The cells were resuspended in AIM V media (Life Technologies #870112dk) with 2% Human AB Sera (HABS) (Valley Biomedical #HP1022) at 2×10⁸ per mL. Twenty-nine mL of AIM V media containing 2% HABS and 1 mL of PBMC cell suspension were added to a T-150 cell culture flask. All cell culture flasks were placed into a single dedicated humidified incubator at 37° C., 5% CO₂ for 2 hours to allow the monocyte precursors to adhere. Following the adherence period, non-adherent cells were removed and the remaining monolayer was washed once with PBS. The adherent cells were replenished with 30 ml of AIM-V per flask supplemented with 800 U/mL recombinant human GM-CSF (Berlex Laboratories, Inc.) and 500 U/mL recombinant human IL-4 (R&D Systems #204-IL/CF), and incubated in humidified incubator at 37° C., 5% CO₂ for 7 days.

After the 7-day culture period, adherent DCs were washed with cold PBS. Ten ml of Dissociation Buffer Enzyme-Free (Life Technologies #13150-016) was added and the DCs were incubate at 4° C. for 10 minutes. The remainder of adherent DC were flushed from the flask and combined with previously harvested DC, then pelleted at 500×g for 10 minutes at 4° C. Following one wash with cold PBS, the cells were checked for number and viability.

### Example 4

### RNA or Peptide Loading & Maturation of Dendritic Cells

Dendritic cells generated as described above are resuspended at 2.5×10⁷ cells per mL of ViaSpan (Belzer UW-CSS, DuPont Pharmaceuticals, Wilmington, Del.). Two hundred μL of the suspension are then placed in a cuvette (Gene Pulser Cuvette, Bio-Rad #165-2086) along with about twenty-five μg of embryonic day 18 (E18) total embryonic RNA, developmental cell RNA, or a stable concentration of peptide extract from a developmental cell and the cells are electroporated (BTX Electro Square Porator #ECM830) at 300 volts for 500 μ seconds. Approximately 1×10⁸ of the electroporated cells are transferred to a T225 flask containing 50 mL of AIM V supplemented with 800 U/mL of recombinant human GM-CSF (Berlex Laboratories, Inc.) and 500 U/ml recombinant human IL-4 (R&D Systems #204-IL/CF), then incubated at 37° C., 5% CO₂ for 1 hour. At the end of the incubation period, the appropriate amount of media with maturation cytokine is added to a final concentration of the cytokines in the maturation cocktail of 10 ng/ml TNF-α (R&D Systems, #210-TA/CF); 10 ng/ml IL-1β (R&D Systems, #201-LB/CF); and 1000 units/ml IL-6 (R&D Systems, #208-IL/CF). The cells were incubated overnight at 37° C. and 5% CO₂.

At the end of the maturation period, the supernatant is removed and placed in chilled 50 ml conical tube on ice. The remaining monolayer is washed with ice cold PBS; combined with supernatant; then pelleted at 500×g for 5 minutes 4° C. The pellet is resuspended in 10 ml of ice cold PBS and kept on ice. Ten to 20 ml of Dissociation Buffer Enzyme-Free (Life Technologies #13150-016) is added and the cells kept at 4° C., and the progress of the cells coming off is monitored every 5 minutes. The flask is washed twice with 10 ml of ice cold PBS, combined with the cells from the dissociation buffer, and pelleted using 500×g. The cells were combined with the other cells, and brought up to 50 ml and counted.

### Example 5

### Preparation of Dendritic Cells for Vaccination

Mature antigen-loaded dendritic cells in a 50 mL conical centrifuge tube and 25 ml of PBS is slowly add, then the cells are pelleted at 200×g for 5 minutes at 22° C. The cells are resuspended in 10 ml of PBS and, using a 2 ml pipette, a 100 μl sample of the suspension is removed and the cells are counted using a hemocytometer and trypan blue as described above. Cells are determined to be ≧70% viable before proceeding. Cells are pelleted at 500×g for 5 min at 22° C. and resuspended at 5×10⁴ cells/mL in 0.9% sodium chloride. The cells are loaded into a 1 cc syringe with 25 G ⅝ gauge needle. Samples are sent for Gram stain and endotoxin testing prior to administration.

### Example 6

### Activation of T Cells

DCs are generated from normal volunteers and subjects with cancer and pulsed with developmental cell peptide extract in AIM-V medium—2% human AB serum for 3 hr at 37° C. Alternatively, for loading with mRNA encoding a plurality of developmental cell antigens, DCs are washed and resuspended in Opti-MEM for electroporation. A suitable concentration of mRNA encoding the plurality of developmental cell antigens per amount of DCs is electroporated with the BTX ECM 830. DCs are washed once with 45 ml of AIM-V medium and autologous responder lymphocytes are added at a 1:10 ratio (DC:T cells). As a source of T lymphocytes, nonadherent (NA) cells are generated from peripheral blood mononuclear cells (PBMCs) from subjects with, e.g., malignant gliomas and normal individuals. Mononuclear cells from peripheral blood are isolated by Ficoll-Hypaque gradient separation (LSM; MP Biomedicals, Solon, Ohio).

Volumes are adjusted to about 2×10⁶ cells/ml and cells are incubated at 37° C. in a humidified atmosphere containing 5% CO₂. After 3 days, an equal amount of AIM-V medium with 2% pooled human AB serum plus IL-2 (10 U/ml) is added and cells are transferred to 24-well plates at a volume of 1 ml/well. Thereafter, every 2-3 days, cells are evaluated for growth and adjusted to about 1×10⁶ cells/ml. After 8 to 11 days of co-culture with pulsed DCs, the T cells are harvested.

### Example 7

### Nanoparticle/Lipid Delivery of Vaccine

Developmental cell RNAs, proteins, or peptide lysates from embryonic or fetal tissues, pluripotent stem cells, or tissue-derived progenitor cells are each complexed with clinical-grade nanoparticle or lipid formulations for delivery in vivo into human subjects. Examples of suitable complexing agents include, but are not limited to, GMP-grade DOTAP & DOTAP:Cholesterol (Boehringer Mannheim, Germany), cGMP grade in vivo-jetPEI™ (Polyplus transfection, Inc. NY, N.Y.), and dendrimer nanoparticles. RNA is precipitated on gold or silver nanoparticles for intradermal delivery by gene gun technology. A suitable ratio of RNAs, proteins, or peptide lysates to complexing transfection agent is mixed in vitro to form peptide or RNA:nanoparticle/liposome complexes and then delivered through intravenous, intradermal, subcutaneous, or intramuscular delivery to human subjects in order to induce an immunologic response against the development cell antigens and shared cancer antigens. Delivery of an adjuvant before, with, and after vaccination with developmental cell antigens is performed (e.g., 150 micrograms of GM-CSF is delivered at vaccine site 24 hrs before, with , and after vaccination).

### Example 8

### Generating Amplified cDNA Template for Continuous RNA Production

To demonstrate that RNA from limiting amounts of sorted cells can be amplified to clinical scale for continuous RNA production for use in various applications (e.g., for loading DCs), amplified mRNA was successfully generated in an FDA-approved clinical grade cell processing facility from as few as 500 CD133⁺ cancer stem cells and CD133⁻ sorted tumor cells. Four housekeeping genes (i.e., 18S, β-actin, GADPH, HPRT) and the CD133 gene were determined every five cycles during amplification using real-time comparative PCR incorporating gene-specific probes and primers spanning joining regions of exons at the 5′ end of the genes for detection of full-length or near full-length amplified cDNA products. No CD133 gene expression was detected in RNA isolated from CD133⁻ cells.

Comparative PCR quantification of CD133 RNA to the individual housekeeping genes are shown in Table 1. The ratio of CD133 RNA detected using 500 cells to 1000 input cells normalized against each of the four housekeeping genes was about 2.

TABLE 1
PCR Quantification of CD133 RNA

| | | Detection of | Ratio: | Ratio: | Ratio: | Ratio: |
|---|---|---|---|---|---|---|
| | # of Sorted | CD133 RNA in | CD133/ | CD133/ | CD133/ | CD133/ |
| Cell | Cells for RNA | 1000/500 | 18S | β-actin | GAPDH | HPRT |
| Fraction | extraction | cells | RNA | RNA | RNA | RNA |
| Patient 179 | 500 | — | 0.00252 | 0.08054 | 0.03271 | 0.91117 |
| CD133(+) | | | | | | |
| Patient 179 | 1000 | 2.0455 | 0.00438 | 0.17263 | 0.07514 | 1.82233 |
| CD133(+) | | | | | | |
| Patient 179 | 500 | not detected | NA | NA | NA | NA |
| CD133(−) | | | | | | |
| Patient 179 | 1000 | not detected | NA | NA | NA | NA |
| CD133(−) | | | | | | |

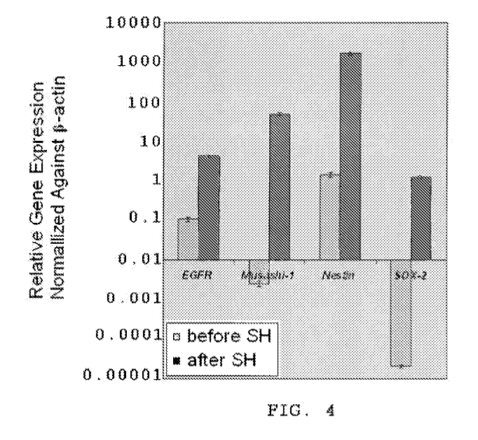

Amplification products were visualized using agarose gel electrophoresis (FIG. 3a). The relative proportions of genes remained constant up to 30 cycles of amplification after initial cDNA library synthesis, after which a gradual decrease of CD133 compared to housekeeping genes was observed. Using 25 cycles of amplification of the cDNA library as a stopping point for RNA synthesis, over 10 μg of template cDNA were generated from 500 CD133⁺ cells in two patient samples tested.

RNA synthesis from 8 μg of template yielded over 980 μg of in vitro transcribed mRNA (IVT-RNA) (FIG. 3b). IVT-RNA was analyzed by gel electrophoresis and demonstrated a range of nucleic acid species from 100 bp surpassing 4.0 kb in length. Larger RNA species at lesser quantities were visible but overexposure of much more abundant smaller RNAs precluded photographic evaluation. These results demonstrate the capacity to amplify RNA from limiting numbers of sorted cells (e.g., CD133⁺ tumor cells) to clinical scale, for example for loading of DCs.

### Example 9

### Enriching for Antigens By Subtractive Hybridization

Optionally removing antigens shared by normal adult tissue may reduce autoimmune risk and enhance immunity against desired target antigens. Enriching for developmental cell antigens can be performed by a number of techniques, for example enriching for nucleic acids encoding developmental cell antigens by subtractive hybridization. For example, Dynabeads® Oligo(dT)25 (Invitrogen Corporation, Carlsbad, Calif.) can be used to produce subtracted cDNA probes for screening and isolation of rare and/or differentially expressed mRNAs. Bead-bound oligo-dT sequence can be used to prime cDNA synthesis to produce a cDNA libraries specific for a particular cell type or tissue.

Cancer stem cell antigens, for example, can be enriched using subtractive hybridization of normal brain RNA and non-cancer stem cell RNA in malignant brain tumor specimens. The protocol for subtractive hybridization and cDNA library synthesis for generating templates for RNA production encoding antigens expressed specifically in developmental cells is performed using, e.g., a commercially available subtractive hybridization kits (Invitrogen Corporation, Carlsbad, Calif.). Subtractive hybridization also is described by e.g., Hansen-Hagge et al., Nucl. Acids Res. 29(4):e20 (2001); Pradel et al., Appl. Env. Microbiol. 68:2316-2325 (2002); and Laveder et al., Nucleic Acids Res. 30(9): e38 (2002), each of which is incorporated herein for its teaching of subtractive hybridization.

For example, target mRNA comprises either total developmental cell or embryonic stem cell RNA; and subtractor mRNA comprises normal RNA from target tissues (i.e. brain) or a pool of normal RNA from human organs to reduce systemic cross-reactivity. After the final hybridization step, the subtracted mRNA from the developmental cells are reverse transcribed to full length cDNA library with PowerScript™ Reverse Transcriptase (Clontech Laboratories, Inc, Mountain View, Calif.) in 10 μL of total reaction volume by priming with modified oligo-dT primer (5′-AAG CAG TGG TAT CAA CGC AGA GTA C(T)₆₄VN-3′) and T7 strand switch primer (5′-AAG CAG TGG TAT CAA CGC AGA GTG GCC ATA TTG GCC rGrGrG/3AmMC6-3′); and amplified using real-time PCR.

The results of amplification of RNA from as few as 500 cancer stem cells from glioblastoma (defined as CD133⁺ tumor cells) is shown in FIG. 4. RNA can be prepared from existing cDNA libraries from developmental cells (i.e. embryonic stem cell cDNA libraries) or prepared from primary developmental cells or established cell lines.

1. A method for eliciting in a subject an immune response to at least one antigen expressed by a cancer cell, the method comprising:
administering to the subject a pharmaceutically acceptable composition comprising a plurality of developmental cell antigens, or nucleic acids encoding the plurality of developmental cell antigens, wherein the pharmaceutically acceptable composition, when administered to the subject, elicits the immune response to the at least one antigen.

2. The method of claim 1, wherein the pharmaceutically acceptable composition is provided in a prophylactically or therapeutically effective amount sufficient to prevent or treat cancer in the subject.

3. The method of claim 1, wherein the plurality of developmental cell antigens, or nucleic acids encoding the plurality of developmental cell antigens, correspond to antigens or nucleic acids encoding antigens from an embryonic stem cell.

4. The method of claim 1, wherein the nucleic acids encoding the plurality of antigens are total RNA or poly A⁺ RNA from a developmental cell.

5. The method of claim 1, wherein the nucleic acids encoding the plurality of antigens are RNAs transcribed from cDNAs from a developmental cell cDNA library.

6. The method of claim 1, wherein the plurality of antigens is a whole cell lysate of a developmental cell.

7. The method of claim 4, wherein the nucleic acids encoding the plurality of antigens comprise RNAs enriched for transcripts (i) not transcribed in a normal postpartum tissue; or (ii) transcribed at a substantially reduced level in the normal postpartum tissue relative to the developmental cell.

8. The method of claim 1, wherein the pharmaceutically acceptable composition further comprises at least one adjuvant, or a nucleic acid encoding the adjuvant.

9. The method of claim 8, wherein the adjuvant is a cytokine selected from the group consisting of: GM-CSF, G-CSF, IL-2, IL-4, IL-7, IL-12, IL-15, IL-21, TNF-\ddot{γ}, and M-CSF.

10. The method of claim 8, wherein the adjuvant comprises incomplete Freund's adjuvant (Montanide ISA 51) or Corynebacterium granulosum P40.

11. The method of claim 1, wherein the developmental cell is xenogeneic to the subject.

12. A pharmaceutically acceptable composition comprising a plurality of antigens expressed by a developmental cell, or nucleic acids encoding the plurality of antigens, wherein the pharmaceutically acceptable composition, when administered to a subject, elicits an immune response against at least one antigen expressed by a cancer cell.

13. The pharmaceutically acceptable composition of claim 12 further comprising a physiologically acceptable carrier, diluent, or excipient.

14. The pharmaceutically acceptable composition of claim 12 further comprising an adjuvant.

15. The pharmaceutically acceptable composition of claim 14, wherein the adjuvant is a cytokine.

16. The pharmaceutically acceptable composition of claim 14, wherein the adjuvant is a cytokine selected from the group consisting of: GM-CSF, G-CSF, IL-2, IL-4, IL-7, IL-12, IL-15, IL-21, TNF-\ddot{γ}, and M-CSF.

17. (canceled)

18. A method for eliciting in a subject an immune response to at least one antigen expressed by a cancer cell, the method comprising:
administering to the subject a composition comprising an effective amount of antigen presenting cells, T-lymphocytes, or both, wherein the antigen presenting cells and T lymphocytes have been sensitized in vitro with a sensitizing-effective amount of a plurality of antigens expressed by a developmental cell, wherein the effective amount of antigen presenting cells, T lymphocytes, or both is sufficient to elicit the immune response to the at least one antigen.

19. The method of claim 19, wherein the antigen presenting cells or T lymphocytes are autologous.

20-32. (canceled)
